(19) United States
(12) Patent Application Publication
(10) Pub. No.: US 2023/0107021 A1
(43) Pub. Date: Apr. 6, 2023

(54) Surgical Guides and Implants Having Periosteum Compressing Projections

(71) Applicant: KLS Martin, LP, Jacksonville, FL (US)

(72) Inventor: Travis Simpson, Jacksonville, FL (US)

(73) Assignee: KLS Martin, LP, Jacksonville, FL (US)

(21) Appl. No.: 17/955,088

(22) Filed: Sep. 28, 2022

Related U.S. Application Data
(60) Provisional application No. 63/249,237, filed on Sep. 28, 2021.

(51) Int. Cl. A61B 17/15

(52) U.S. Cl. CPC A61B 17/15; A61B 2034/108

(57) ABSTRACT

A bone osteotomy guide or bone plate fixation implant whose anterior surface has a plurality of projecting members that are adapted to compress but not cut into the periosteum when positioned and affixed to the bone. Preferably, the projecting members are present as pointed or tapered structures, such as cones or pyramids, wherein the tips of the projecting members are blunt, truncated or rounded. The heights, widths, shapes, numbers and densities of projecting members on a given guide or fixation implant may vary. Because the projecting members compress the periosteum, the tips of the projecting members define a three-dimensional virtual surface that better conforms to the actual surface topography of the bone.

### BACKGROUND OF THE INVENTION

This invention relates generally to osteotomy cutting and drilling guides and bone fixation implants used for example in bone reconstruction, orthognathic surgery or related bone treatments involving osteotomies or bone repair, and in particular to such guides and fixation implants that are patient-specific, i.e., customized, wherein the inner surface of the guides and implants are shaped and configured based on 3-dimensional computer-aided design to conform to the surface topography of the underlying bone. The invention also relates to the method of utilizing the guides and implants.

In some circumstances, a bone may be fractured or otherwise damaged to the extent that a rigid implant must be affixed to the bone in order for it to heal properly, such as for example a fractured femur. In other circumstances, referred to as orthognathic surgery, osteotomies may need to be performed wherein segments of bone are cut or removed in order to allow a portion of the bone to be reattached in a more desirable orientation, such as for example due to malformations of the jaw or maxilla. The word “osteotomy” means the division or excision of bone.

As an example, orthognathic surgery of the jaw and face is surgery designed to correct conditions related to structure, growth, sleep apnea, TMJ disorders, malocclusion problems owing to skeletal disharmonies, or other orthodontic problems that cannot be easily treated with braces. During such surgery, bone is cut to create non-mobile or base bone segment(s) and mobile or separated bone segment(s), and the mobile bone segment(s) are repositioned and realigned to correct a dentofacial or similar deformity, with bone plates or implants used to fix the detached or mobile bone segments to the non-mobile bone segments in the desired post-osteotomy orientation. The osteotomies may be performed on the maxilla (e.g., a LeFort I), the mandible (e.g., a sagittal split), or the chin (e.g., a genioplasty).

Early techniques utilized stock or standardized fixation plates that had to be manipulated (i.e., cut or bent) by the surgeon to provide a better fit onto the bone surface topography. Such techniques resulted at best in approximations to the bone surface topography.

Modern orthognathic surgery makes use of computer-aided design and manufacturing techniques whereby surgeons and technicians create pre-osteotomy and post-osteotomy virtual 3-D models of a patient’s bone structure topography. The virtual pre-osteotomy 3-D model is dproduced using various electronic scanning techniques and shows the current configuration of the bone structure before corrective surgery. The surgeon along with technicians then virtually manipulate the pre-osteotomy model to produce the desired post-osteotomy configuration and orientation for the corrected bone structure. One or more virtual guides for cutting the osteotomy or marking the location of the osteotomy on the maxilla, mandible or chin, as well as for drilling holes to receive bone fastening screws, are created within the computer system, and actual guides are then manufactured based on the specifications of the virtual guides. Likewise, one or more virtual fixation bone plates are then configured and actual fixation bone plates are produced from the specifications, such that when the actual fixation bone plates are attached to the non-mobile bone segment and the re-positioned mobile or detached bone segment after the osteotomy, the bone segments will be properly positioned relative to each other in the desired post-osteotomy configuration.

The guide is designed to conform to the pre-osteotomy surface topography or configuration of the patient’s bone structure, the inner or anterior surface of the guide matching the surface topography of the bone such that the surgeon can easily position the guide in the proper location for making the osteotomy cuts. In one type of procedure, the surgeon then uses the guide to mark the location for the osteotomy and either marks or drills holes through apertures in the guides, the holes being properly positioned to receive the bone screws used to fasten the fixation implants to the bone segments with the bone segments positioned in the desired post osteotomy relationship. The guide is then removed and the osteotomy cuts are then made. Alternatively, rather than using the guide for marking purposes, the guide may be produced with physical structures, e.g., edges or slits, to guide the osteotomy saw during the severing of the bone. The fixation bone plates are produced such that a first anterior portion of the plate conforms to the surface topography of the non-mobile bone segment and a second anterior portion of the plate conforms to the surface topography of the mobile bone section. A transition portion connects the first and second conforming portions, such that the positioning and orientation of the second conforming portion results in proper relocation and orientation of the mobile bone segment.

While this computer-aided orthognathic surgery method and devices is an improvement over the earlier techniques and devices, proper positioning of the guide and fixation bone plates is still problematic, due to the presence of compressible periosteum material, the dense layer of vascular connective tissue enveloping the bone, which has not been thoroughly or consistently removed by the surgeon prior to use of the guide and/or implant. The periosteum layer remaining on the bone will vary in average thickness from fractions of a millimeter to multiple millimeters depending on the surgeon. In many instances surgeons do not strip or remove any of the periosteum as this may hinder healing. Even stripping the periosteum usually results in variations in periosteum thickness at multiple discrete locations across the site. The presence of the periosteum makes it difficult to properly fit the guides and then fixation implants onto the bone, which is especially problematic in orthognathic surgery where precise translation of the mobile bone segment into the post-osteotomy orientation relative to the non-mobile bone is the goal.

One solution that has been utilized in the art is to provide the anterior surfaces of the guides and fixation implants with an arbitrary offset of 1.0 millimeter or less to account for the presence of the periosteum on the bone. But this is merely an estimation and does not take into account the variations in periosteum thickness across the site.

It is an object of this invention to address the periosteum problem by providing guides and fixation implants with improved anterior surfaces, the surfaces comprising a plurality of projecting members that are adapted to compress the periosteum. Preferably, the projecting members are present as pointed or tapered structures, such as cones or pyramids, wherein the tips of the projecting members are blunted, rounded, truncated, etc. In this manner each of the projecting members compress the periosteum but do not cut into or pierce the periosteum. The heights, widths, shapes, numbers and densities of projecting members on a given guide or fixation implant may vary. Because the projecting member compress the periosteum, the tips of the projecting members define a three-dimensional “virtual surface” that better conforms to or mimics the actual surface topography of the bone than the anterior surface of the guide or implant.

### SUMMARY OF THE INVENTION

In brief summary, the invention in general is an osteotomy guide or a bone fixation implant whose anterior surface comprises a plurality of projecting members that are adapted to compress but not cut into the periosteum when positioned and affixed to the bone. Preferably, the projecting members are present as pointed or tapered structures, such as cones or pyramids, wherein the tips of the projecting members are blunted, truncated or rounded. Bone screw receiving apertures are provided such that the guides and fixation implants may be secured to the bone using standard bone screws. The heights, widths, shapes, numbers and densities of projecting members on a given guide or fixation implant may vary, and will vary when the guide or implant is designed and produced using computer-aided design and manufacturing techniques. Because the projecting members compress the periosteum, the tips of the projecting members define a three-dimensional, “virtual surface” that better conforms to, mimics or matches the actual surface topography of the bone. The guides and fixation implants may present a solid body member, or alternatively openings through the body may be provided between the projecting members such that the guide or fixation implant presents a mesh-like appearance.

### BRIEF DESCRIPTION OF THE DRAWINGS

FIG. 1 illustrates a representative embodiment of an osteotomy guide member comprising a plurality of projecting members extending from a solid body, a plurality of marking or cutting slots, and a plurality of bone screw receiving openings.

FIG. 2 illustrates a representative embodiment of an implant member comprising a plurality of projecting members extending from an apertured body.

FIG. 3 is a cross-sectional view of FIG. 2 taken along line 3-3 illustrating the virtual surface defined by the projecting members.

### DETAILED DESCRIPTION OF THE INVENTION

It is to be understood that the drawings are presented for illustrative, enabling and descriptive purposes. The drawings present a representative embodiment of the invention and are not intended to be limiting as to the scope and definition of the invention. As used herein the term “anterior surface” defines the surface that faces the bone surface when the guide or implant is positioned on the bone.

The invention in general is an osteotomy guide 10 or a bone fixation implant 20 whose anterior surface 11/21 comprises a plurality of projecting members 12/22 that are adapted to compress but not cut into the periosteum when positioned and affixed to a bone, whether the affixation is intended to be permanent or long term in the case of fixation implants 20 or temporary in the case of osteotomy guides 10. Preferably, the projecting members 12/22 are configured and shaped as pointed, tapered or mounded structures, such as cones or pyramids, wherein the tips 13/23 of the projecting members 12/22 are blunted, truncated or rounded. Bone screw receiving apertures 14/24 are provided such that the guides 10 and fixation implants 20 may be secured to the bone using standard bone screws. The heights, widths, shapes, numbers and densities of projecting members 12/22 on a given guide 10 or fixation implant 20 may vary. Most preferably, the guides 10 and implants 20 are customized to each particular patient using computer-aided design and manufacturing techniques that are well known in the art. Because the projecting members 12/22 compress the periosteum, the tips 13/23 of the projecting members 12/22 define a three-dimensional virtual surface 30 (shown in FIG. 3) that better conforms to, mimics or matches the actual surface topography of the bone. The guides 10 and fixation implants 20 may be constructed as a solid body member, or openings through the body may be provided between the projecting members 13/23 such that the guide 10 or fixation implant 20 presents a mesh-like appearance.

Standard guides 10 and fixation implant members 20 not configured using 3-D computer-aided design may be improved by providing projecting members 12/22 on the anterior surfaces of the fixation implant 20 or guide 10, as compression of the periosteum by the projecting members 12/22 will improve the fit of the guide 10 or implant 20. Most preferably, however, the fixation implants 20 and guides 10 are configured using 3-D computer-aided design, especially in the case of orthognathic surgeries, as the implants 20 and guides 10 so produced are customized for optimized matching of the bone surface topography.

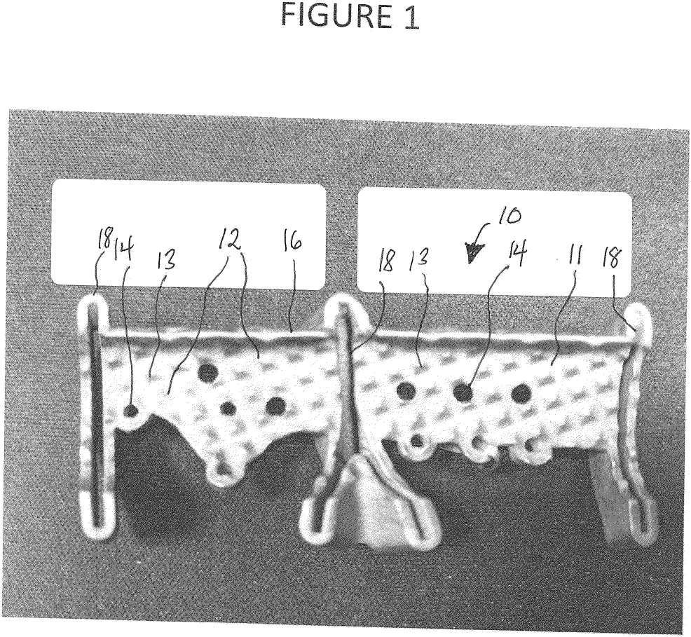

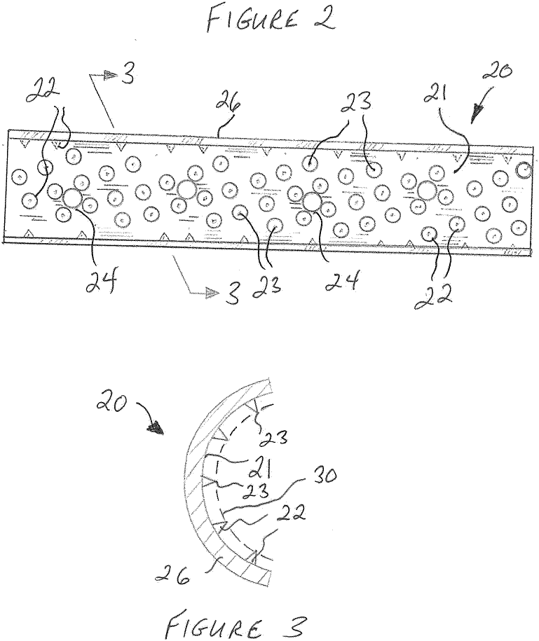

FIG. 1 illustrates an embodiment of an osteotomy guide 10 comprising a plurality of projecting members 12 extending from the anterior surface 11 of the main body 16, a plurality of marking or cutting slots 18, and a plurality of bone screw receiving apertures 14. A representative bone fixation implant 20, as shown in FIG. 2, comprises a plurality of projecting members 22 extending from the anterior surface 21 of the main body 26, and a plurality of bone screw receiving apertures 24 would lack the marking or cutting slots 18 of the guide 10.

As seen in FIG. 1, the anterior surface 11 of the guide 10 comprises a plurality of pyramid-shaped projecting members 12, shown as being four-sided in this embodiment. The tips 13 of the projecting members 12 are rounded, truncated or blunt, as it is desirable that the tips 13 not pierce or cut into the periosteum when the guide 10 is fitted onto the bone.

FIG. 2 illustrates a representative embodiment of a bone fixation implant 20 comprising a plurality of cone-shaped projecting members 22. The tips 23 of the projecting members 22 are rounded, truncated or blunt, as it is desirable that the tips 23 not pierce or cut into the periosteum when the implant 20 is fitted onto the bone.

As shown with reference to the implant of FIG. 2, FIG. 3 illustrates the combination of the projecting member tips 13 defining an artificial three-dimensional virtual surface 30 that is configured to match the surface topography of the bone at the site. While it is possible to estimate this best fit configuration for the location, spacing, shape and size of the projecting members 12/22, it is most preferable that the projecting members 12/22, along with the other portions of the guide 10 or implant 20, be determined using 3-D computer-aided design and manufacture as discussed above. With the guide 10 or implant 20 properly located on the bone, bone screws are inserted into the bone screw receiving apertures 14/24 and tightened for temporary or permanent affixation. As the screws are tightened to affix the guide 10 or implant 20 to the bone, the projecting members 12/22 compress the periosteum and provide a more precise and secure fit of the guide 10 or implant 20 onto the bone. For the guide 10, the affixation is temporary and the guide 10 is removed from the bone and periosteum after the osteotomies are marked or cut. For a fixation implant 20, the implant 20 will remain affixed to the bone and periosteum.

FIGS. 1 and 2 illustrate an embodiment for an osteotomy guide 10 or implant 20 comprising a plurality of projecting members 12/22 extending from a substantially solid main body 16/26, but alternatively the body of a guide 10 or fixation implant 20 may be provided with apertures or open areas separate from and in addition to the bone screw receiving apertures 14/24. With this structure, the body of the guide 10 or implant 20 presents a mesh-like configuration providing space for expansion of the periosteum material not compressed by the projecting members 12/22, while also providing open areas visible to the surgeon. The open areas may also be beneficial for better or faster healing and for reducing infection.

The methodology for design, manufacture and use of the guides 10 and implants is best illustrated in terms of orthognathic surgery. The first step involves the use of computer-aided design and manufacturing techniques whereby surgeons and technicians create pre-osteotomy and post-osteotomy virtual 3-D models of a patient’s bone structure topography. The virtual pre-osteotomy 3-D model is produced using various electronic scanning techniques and shows the current configuration of the bone structure before corrective surgery. The surgeon along with technicians then virtually manipulate the pre-osteotomy model to produce the desired post-osteotomy configuration and orientation for the corrected bone structure. One or more virtual guides for cutting the osteotomy or marking the location of the osteotomy on the maxilla, mandible or chin, as well as for drilling holes to receive bone fastening screws, are created within the computer system. The topography of the periosteum is noted and the location, height, and other structural characteristics of the projections 12 are configured as necessary to define the virtual surface 30 and to account for the variations in the thickness of the periosteum at multiple points. Actual guides 10 having projecting members 12 are then manufactured based on the specifications of the virtual guides. In the same manner, one or more virtual fixation bone plates are then configured and actual fixation bone plate implants 20 are produced from the specifications, such that when the actual fixation bone plate implants 20 are attached to the non-mobile bone segment and the re-positioned mobile or detached bone segment after the osteotomy, the bone segments will be properly positioned relative to each other in the desired post-osteotomy configuration

Once the osteotomy guide 10 has been designed and manufactured, the surgeon positions the guide 10 on the bone and affixes the guide 10 by tightening bone screws inserted through the bone screw receiving apertures 14. The bone screws are tightened so as to draw the guide toward the bone, which results in compression of the periosteum where contacted by the tips 13 of the projecting members 12. The portions of the periosteum adjacent the tips 12 may contact the inner surface of the main body, depending on the height of the projecting members 12 and the thickness of the periosteum at each projecting member 12. Once the bone is marked or cut, the guide 10 is removed by unscrewing the bone screws. After completion of the osteotomy and positioning of the bone segments, the fixation implant 20 is affixed to the bone segments using bone screws inserted through the bone screw receiving apertures 24. As before, the tips 23 of the projecting members 22 compress the periosteum, thereby providing a more secure and accurate fixation to the bone.

It is understood that equivalents and substitutions for certain elements set forth above may be obvious to those of skill in the art, and therefore the true scope and definition of the invention is to be as set forth in the following claims.

I claim:

1. An osteotomy guide comprising:
a main body, osteotomy marking or cutting slots, bone screw receiving apertures, and an anterior surface;
projecting members positioned on the anterior surface, said projecting members having tips that are blunted, rounded or truncated;
the projecting members characterized such that with the osteotomy guide affixed onto a bone covered totally or partially with a periosteum, the tips of the projecting members compress but do not pierce the periosteum.

2. The osteotomy guide of claim 1, wherein the projecting members are pyramid-shaped.

3. The osteotomy guide of claim 1, wherein the projecting members are cone-shaped.

4. The osteotomy guide of claim 1, wherein the location, size and shape of the projecting members are determined using computer-aided design and manufacturing techniques whereby surgeons and technicians create pre-osteotomy and post-osteotomy virtual 3-D models of a patient’s bone structure topography, such that the tips of the projecting members define a virtual surface approximating the surface topography of the bone.

5. The osteotomy guide of claim 4, wherein the tips of the projecting member define a virtual surface conforming to the surface topography of the bone.

6. An osteotomy guide comprising:
a main body, osteotomy marking or cutting slots, bone screw receiving apertures, and an anterior surface;
projecting members positioned on the anterior surface, said projecting members having tips that are blunted, rounded or truncated.

7. The osteotomy guide of claim 6, wherein the projecting members are pyramid-shaped.

8. The osteotomy guide of claim 6, wherein the projecting members are cone-shaped.

9. The osteotomy guide of claim 6, wherein the location, size and shape of the projecting members are determined using computer-aided design and manufacturing techniques whereby surgeons and technicians create pre-osteotomy and post-osteotomy virtual 3-D models of a patient’s bone structure topography, such that the tips of the projecting members define a virtual surface conforming to the surface topography of a bone.
